(19) United States
(12) Patent Application Publication
(10) Pub. No.: US 2019/0259470 A1
(43) Pub. Date: Aug. 22, 2019

(54) ARTIFICIAL INTELLIGENCE PLATFORM FOR PROTEIN ENGINEERING

(71) Applicant: Protabit LLC, Pasadena, CA (US)

(72) Inventors: Barry D. Olafson, Altadena, CA (US); Paul M. Chang, Pasadena, CA (US); Connie Y. Wang, Los Angeles, CA (US); Wesley Aaron Field, Los Angeles, CA (US); Shu-Ching Ou, San Gabriel, CA (US); Mary L. Ary, Encino, CA (US)

(21) Appl. No.: 16/277,294

(22) Filed: Feb. 15, 2019

Related U.S. Application Data
(60) Provisional application No. 62/632,169, filed on Feb. 19, 2018.

(51) Int. Cl. G16B 20/50; G16B 40/00; G06N 20/10; G16B 30/00; G16B 50/10

(52) U.S. Cl. CPC G16B 20/50; G16B 40/00; G16B 50/10; G16B 30/00; G06N 20/10

(57) ABSTRACT

An artificial intelligence platform, a database for storage and analysis of protein engineering data, and a deposition tool used to parse and store protein engineering data. Specifically, machine learning processes are used for processing large amounts of protein mutation information in order to engineer proteins with specific functions.

### CROSS-REFERENCE TO RELATED APPLICATIONS

This application claims the benefit of U.S. Provisional Patent Application 62/632,169 filed on Feb. 19, 2018, which is incorporated herein by reference. This application is related to PCT/US2019/018221, filed on Feb. 15, 2019, which is incorporated herein by reference.

### GOVERNMENT LICENSE RIGHTS

This invention was made with government support under R44GM117961 awarded by NIH; R44GM113542 awarded by NIH; and IIP-1534743 awarded by NSF.

The government has certain rights in the invention.

### TECHNICAL FIELD

The present disclosure relates generally to an artificial intelligence platform, a database for storage and analysis of protein engineering data, and a deposition tool used to parse and store protein engineering data. Specifically, the disclosure relates to machine learning processes used for processing large amounts of protein mutation information in order to engineer proteins with specific functions.

### BACKGROUND

Recent advances in gene synthesis, microfluidics, deep sequencing, and microarray techniques have greatly facilitated the ability of researchers to engineer variant protein sequences. Thousands or even millions of sequence variants can now be generated and screened in an ultrahigh-throughput fashion. This rapid generation of large sets of mutational data has enabled comprehensive mappings between protein sequence and function for properties such as stability, binding affinity, and catalytic activity. Deep mutational scanning approaches have been used to study protein fitness landscapes, discover new functional sites, and engineer proteins with new and improved properties. Many groups are now using these techniques to generate large amounts of protein engineering (PE) data—a trend that is expected to grow in the future.

The field of PE thus appears to be entering into a state reminiscent of the early days of structure determination and genome sequencing, poised for the development of transformation new technologies based on PE. Unfortunately, there is no database for depositing, describing, storing, searching, query, managing and analyzing that is customized for complex and massive protein data. There is no elegant means of sharing the data and analysis with collaborators. There are also no tools to take advantage of the vast amounts of mutational data to intelligently design new proteins based on wanted function. The absence of these functionalities as applied to complex proteins creates obstacles for development of new PE technologies in a wide range of fields. This disclosure addresses these needs and presents a database, data acquisition tools, data analysis tools, and an artificial intelligence platform towards the development of proteins based on wanted function.

Protein engineering plays a key role in advancing biotechnology and medicine. The manipulation of a protein's properties by modifying the underlying protein sequence is one of the most powerful engineering approaches that can be applied to problems in human health. Proteins are increasingly serving as drugs and drug delivery devices. In the last decade, antibodies and other protein therapeutics have moved to the forefront of drug discovery, comprising 7 of the top 10 highest revenue drugs. Most of these protein drugs have been engineered. Engineering can dramatically improve important properties such as efficacy, binding affinity, and serum half-life; it can decrease toxicity and immunogenicity, and can even produce entirely new specificities and modes of action. Engineered proteins have also proven useful as research tools. For example, engineering can lead to protein variants that are more amenable to biophysical characterization or are better suited for the high-throughput screening of small molecule inhibitors or agonists.

Since protein sequence space is vast, multiple properties must be optimized. The main challenges scientists face in protein drug development are two-fold. First, the vastness of protein sequence space means that an astronomically large number of mutations can be explored to find a sequence with the desired properties. Experimental and computational approaches must be applied to focus the mutations in regions that are more likely to produce the desired results. Second, in addition to engineering for proper function or activity, such as binding to a drug target or deactivating disease-causing biomolecules, other properties like expression level, solubility, and serum half-life can also be maintained or improved to produce an effective protein therapeutic.

The engineering approaches currently used to develop protein therapeutics include rational design, directed evolution (DE), and computational protein design (CPD), and all have significant limitations. Rational design typically considers only a handful of mutations since they must be manually visualized and analyzed. This requires a 3D model of the protein, which is often not available. Efforts to determine the 3D structure can take years and are not guaranteed to be successful. Rational approaches also depend on some level of understanding of how the protein functions, which is often unclear or incomplete. DE experiments have been a mainstay in protein engineering but suffer from the need for high-throughput screens or selections, which may not be available for the protein or property of interest. When these assays can be employed, DE is very good at finding useful variants, but mutations are typically limited to those that are close to the starting sequence. Since DE only explores this limited portion of the sequence landscape, it can miss out on the viable mutations that require large jumps in sequence space. CPD methods suffer from a lack of accuracy in the predicted sequences. Better score functions and search algorithms are needed, and although progress is being made, it is not as fast as the pharmaceutical industry would like and at much computational expense. In general, CPD methods also require expert users to run the calculations and analyze the results.

### SUMMARY

In general, in one aspect, the disclosure relates to a system for engineering proteins based on mutational data comprising: a processor; a storage repository comprising: a database comprising: a plurality of full length mutant protein sequences, each full length mutant protein sequence comprising a string representing an amino acid sequence; and a plurality of characteristic data sets, wherein each characteristic data set is associated with one of the full length mutant protein sequences and wherein the characteristic data sets includes data from assays done with the protein of the respective full length mutant protein sequence. In a specific embodiment, the system includes an AI Platform comprising: computer executable instructions for execution by the processor comprising: generating an AI training set comprising a plurality of full length mutant protein sequences from the database; encoding an input tensor comprised of the amino acid sequences of the plurality of full length mutant protein sequences from the AI training set; encoding an output tensor comprised of one or more of the plurality of characteristic data associated with the plurality of full length sequences from the AI training set; and, generating a machine learning model using a machine learning framework configured to input the input tensor and the output tensor, and to generate the machine learning model. In some embodiments, the AI Platform can resides in the storage repository, in memory or in both. In some embodiments, encoding the input tensor comprises encoding individual amino acid characteristics, partial sequences characteristics, or local behavior characteristics. In some embodiments, the data from assays comprise experimental assay type, numerical value obtained for the assay, and units associated with the numerical value. Characteristic data can also include equations and values derived from using the equations on assay data. The characteristic data sets additionally comprise protein structure data. In some embodiments, encoding the input tensor depends on the protein structure data. The input tensors can comprise one or more of charge, hydrophobicity, and volume associated with amino acids in the amino acid sequences of the plurality of full length mutant protein sequences from the AI training set. The charge hydrophobicity and volume can also be calculated for a partial sequence of the protein sequence for use in the input tensors, for example. In some embodiments, the machine learning framework comprises one or more of a neural network, genetic algorithm, decision tree, gradient boosting, and support vector machines. Additionally, the computer executable instructions can further comprise instructions for: receiving a protein identifier and protein functional data; matching the identifier to one or more full length mutant protein sequences stored in the database; creating the AI training set with the matched full length mutant protein sequences; generating a plurality of synthetic sequences; applying the machine learning model to the plurality of synthetic sequences to generate predicted protein functional data for each synthetic sequence; and, outputting one or more of the synthetic sequences and associated predicted protein functional data. In some embodiments, the computer executable instructions can further comprise generating a subset of synthetic sequences in which the predicted protein functional data is within a predetermined range of the received protein functional data. In some embodiments, the synthetic sequences are generated by random mutation or by a computationally designed combinatorial library. The received protein functional data, the characteristic data set, or both can comprise one or more of the following: Activity, Catalytic efficiency (k_{cat}/Kₘ), Catalytic rate constant (k_{cat}), Count/Number, EC50, Energy, Enrichment, Epistasis, Fitness, IC50, Inhibition constant (Kᵢ), Maximal rate (Vₘₐₓ), Michaelis constant (Kₘ), Relative activity, Specific activity, Association constant (Kₐ), Binding affinity, Count/Number, Dissociation constant (K_{d}), ELISA, Energy, Enrichment, Enthalpy of binding (ΔH), Entropy of binding (ΔS), Epistasis, Fitness, Frequency of occurrence, Gibbs free energy of binding (ΔG), Inhibition constant (Kᵢ), Rate constant of association (kₒₙ), Rate constant of dissociation (k_{off}), Concentration, Energy, Enrichment, Frequency of occurrence, Minimum inhibitory concentration (MIC), Yield, Antimicrobial resistance, Energy, Enrichment, Frequency of occurrence, Optical density (OD), Bioavailability, EC50, Half-life (t_{1/2}), IC50, Immunogenicity, Toxicity, Concentration, Energy, Fractional increase in solubility, Insoluble fraction, Oligomerization state, Soluble fraction, Energy, Frequency of occurrence, Relative activity, Relative affinity, Relative k_{cat}, Relative k_{cat}/Kₘ, Relative K_{d}, Brightness, Emission wavelength (λₑₘ), Energy, Excitation wavelength (λₑₓ), Extinction coefficient, Fluorescence intensity, Maturation half-time, Photobleaching half-time, pKa, Quantum yield, Constant pressure heat capacity of unfolding (ΔCₚ), Count/Number, Denaturant concentration at midpoint of unfolding transition (Cₘ), Energy, Enthalpy of unfolding (ΔH), Entropy of unfolding (ΔS), Equilibrium constant (K), Gibbs free energy of folding/unfolding (ΔG), Melting temperature (Tₘ), Rate of folding (k_{F}), Rate of unfolding (k_{U}), Slope of chevron plot (m), Slope of the denaturant unfolding curve/cooperativity value (m), Temperature of maximum stability, Thermal tolerance, ß-Tanford value, and Φ-value. In some embodiments, the protein identifier is a name or a full length protein sequence. In specific embodiments, matching comprises comparing the full length protein sequence of the protein identifier to full length mutant protein sequences in the database and returning a match when the sequences are at least 20%, 30%, 40%, 50%, 60%, 70%, 80%, 90%, 95%, or more than 99% similar. The percent similarity could also be a user entered value. For example, the user could enter 45% and protein mutant sequences with greater than 45% match would be returned. In another embodiment, the storage repository further comprises computer executable instructions for execution by the processor comprising: receiving a protein identifier; matching the identifier to one or more full length mutant protein sequences stored in the database; and, outputting the matched full length mutant protein sequences and the data from assays associated with each matched full length mutant protein sequence.

Another general embodiment is a computer-executed method of engineering proteins, the method comprising: storing a plurality of full length mutant protein sequences and a plurality of characteristic data sets in a database, wherein each characteristic data set is associated with one of the protein mutant sequences, wherein each protein mutant sequence comprises a string representing a sequence of amino acids, and wherein the characteristic data sets include data from assays done with the protein of the respective full length mutant protein sequence; receiving a protein identifier and protein functional data; matching the protein identifier to one or more full length mutant protein sequences stored in the database; generating an AI training set with the matching full length mutant protein sequences; training a machine learning model using the AI training dataset; employing the machine learning model to design one or more synthetic protein sequences and calculate each synthetic proteins predicted functional data; and outputting the one or more synthetic protein sequences and predicted functional data. In some embodiments, the data from assays comprises one or more of experimental assay type, numerical value obtained for the assay, units associated with the numerical value, derived values dependent on other experimental values. In some embodiments, wherein the machine learning model comprises one or more of a neural network, genetic algorithm, decision tree, gradient boosting, and support vector machines. In specific embodiments, matching comprises comparing the full length protein sequence of the protein identifier to the full length mutant protein sequences in the database and returning a match when the sequences are at least 20%, 30%, 40%, 50%, 60%, 70%, 80%, 90%, 95%, or more than 99% similar. The percent similarity could also be a user entered value. For example, the user could enter 45% and protein mutant sequences with greater than 45% match would be returned. In some embodiments, the characteristic data set, the received protein functional data, or both comprises one or more of the following: Activity, Catalytic efficiency (k_{cat}/Kₘ), Catalytic rate constant (k_{cat}), Count/Number, EC50, Energy, Enrichment, Epistasis, Fitness, IC50, Inhibition constant (Kᵢ), Maximal rate (Vₘₐₓ), Michaelis constant (Kₘ), Relative activity, Specific activity, Association constant (Kₐ), Binding affinity, Count/Number, Dissociation constant (K_{d}), ELISA, Energy, Enrichment, Enthalpy of binding (ΔH), Entropy of binding (ΔS), Epistasis, Fitness, Frequency of occurrence, Gibbs free energy of binding (ΔG), Inhibition constant (Kᵢ), Rate constant of association (kₒₙ), Rate constant of dissociation (k_{off}), Concentration, Energy, Enrichment, Frequency of occurrence, Minimum inhibitory concentration (MIC), Yield, Antimicrobial resistance, Energy, Enrichment, Frequency of occurrence, Optical density (OD), Bioavailability, EC50, Half-life (t_{1/2}), IC50, Immunogenicity, Toxicity, Concentration, Energy, Fractional increase in solubility, Insoluble fraction, Oligomerization state, Soluble fraction, Energy, Frequency of occurrence, Relative activity, Relative affinity, Relative k_{cat}, Relative k_{cat}/Kₘ, Relative K_{d}, Brightness, Emission wavelength (λₑₘ), Energy, Excitation wavelength (λₑₓ), Extinction coefficient, Fluorescence intensity, Maturation half-time, Photobleaching half-time, pKa, Quantum yield, Constant pressure heat capacity of unfolding (ΔCₚ), Count/Number, Denaturant concentration at midpoint of unfolding transition (Cₘ), Energy, Enthalpy of unfolding (ΔH), Entropy of unfolding (ΔS), Equilibrium constant (K), Gibbs free energy of folding/unfolding (ΔG), Melting temperature (Tₘ), Rate of folding (k_{F}), Rate of unfolding (k_{U}), Slope of chevron plot (m), Slope of the denaturant unfolding curve/cooperativity value (m), Temperature of maximum stability, Thermal tolerance, ß-Tanford value, and Φ-value.

These and other aspects, objects, features, and embodiments will be apparent from the following description and the appended claims.

### BRIEF DESCRIPTION OF THE DRAWINGS

The drawings illustrate only example embodiments and are therefore not to be considered limiting in scope, as the example embodiments may admit to other equally effective embodiments. The elements and features shown in the drawings are not necessarily to scale, emphasis instead being placed upon clearly illustrating the principles of the example embodiments. Additionally, certain dimensions or positionings may be exaggerated to help visually convey such principles. In the drawings, reference numerals designate like or corresponding, but not necessarily identical, elements.

FIG. 1 illustrates an example ProtaBank database design. Acronyms are AWS, Amazon Web Services; and RDS, Relational Database Services.

FIG. 2 shows an example ProtaBank database schema.

FIGS. 3A and 3B are screenshots of the web interface when using ProtaBank search and analysis tools. FIG. 3A shows a text-based search for “ubiquitin” which returns a sortable table containing all studies with ubiquitin in the protein name or study title. FIG. 3B shows the analysis page for a study on β-lactamase.

FIG. 4 illustrates tools for identifying and analyzing closely related mutants of Gβ1 in ProtaBank. FIG. 4A shows a BLAST search of the ProtaBank database for wild-type Gβ1. The heat map shows the frequency of each residue at each position. The wild-type residue is shown in white. FIG. 4B is a histogram showing the number of sequences found at each mismatch level (Count), where the number of mismatches is the number of mutations needed to go from a given mutant sequence to the search sequence.

FIG. 5 is a graph which plots of Cm vs. Tm for Gβ1 data. A plot of all Gβ1 mutant sequences for which both a Tm and Cm were measured (circles) gives a moderate correlation (r=0.45, dotted line). If only data obtained under similar assay conditions is included (restricting Cm data to guanidinium chloride denaturation, pH 5-7, 20-30° C., and Tm data to pH 5-7, no denaturant added) (triangles), a very strong correlation (r=0.80, solid line) between these two measures of stability is observed.

FIGS. 6A and 6B are graphs comparing predicted with experimentally measured ΔΔG values in ProtaBank. The ΔΔG predictor values (ΔΔG_{screen}) were plotted against experimental ΔΔG values reported in the literature (ΔΔGₗᵢₜₑᵣₐₜᵤᵣₑ). FIG. 6A is a graph of an unfiltered search of ProtaBank database identifying 343 mutant sequence pairs with both predicted and experimental ΔΔG values. FIG. 6B is a graph showing a search filtered by the mutations and background sequences from the Olson et al. study yielding 82 pairs, reproducing their data.

FIG. 7 compares fitness and proximity to the binding site for Gβ1 point mutants. The ProtaBank visualizer was used to map the Olson et al. fitness data to the Gβ1 structure and make the two images shown here. Gβ1 is displayed bound to the Fc domain (PDB ID: 1FCC). In FIG. 7A, the Gβ1 backbone is shaded by median deviation from the wild-type value, with large deviations in blue, medium in white, and small to no deviations in red. In FIG. 7B, the backbone is shaded by proximity to the binding partner. The structural analysis shows that most of the Gβ1 residues near the binding interface are particularly sensitive to mutation.

FIG. 8 is a screen shot of the ProtaBank data deposition form for data in a mutant library.

FIG. 9 is a screen shot of the results obtained using “Identify and analyze sequence mutations” tool to do BLAST search on WT Gβ1 showing assays by property table.

FIGS. 10A and 10B are illustrations of layouts for an example AI Platform integrated with the ProtaBank. FIG. 10A illustrates how the ProtaBank AI Platform integrates input data from the ProtaBank mutation database (upper left), which provides information such as sequences, assays, and sequence features through an API, and the PDB structure database (lower left), which provides information such as coordinates, B-factors, and symmetries, with the use of a chosen ML framework (middle left) into a ProtaBank Machine Learning Module (PMLM) to encode, normalize and featurize the data into numerical tensors/arrays. FIG. 10B illustrates examples of how the AI Platform can support several ML frameworks to provide algorithm flexibility using the NumPy, SciPy, and pandas data formats as a common language.

FIG. 11 is a block diagram of an example computer system used to design proteins.

FIG. 12 illustrates a ProtaBank's AI Platform method used to train a model that predicts solubility and fitness for TEM-1 β-lactamase mutants based on deep mutational scanning data deposited in ProtaBank.

FIG. 13 shows a graph of model performance (as measured by mean squared error (MSE)) over the course of the training process using an 80/20 training validation split for the method of FIG. 12.

FIG. 14 shows that the distribution of predicted solubility values (validation set) closely matches the distribution of experimentally measured values for the method of FIG. 12.

FIG. 15 shows the solubility/fitness landscape for mutants in ProtaBank for the method of FIG. 12.

FIG. 16 shows how the trained model can be applied to trial sequences to predict protein properties for the method of FIG. 12.

FIG. 17 shows the predicted vs. measured graph for the validation set after training using green fluorescence protein, showing a high degree of congruence and bimodal distribution between relatively functional and nonfunctional mutations.

### DETAILED DESCRIPTION OF EXAMPLE EMBODIMENTS

The present disclosure relates generally to a database, herein referred to as ProtaBank, for the storage and analysis of protein engineering data, such as protein mutational information. The present disclosure also relates to a deposition tool for use with the database and an AI platform for protein engineering. ProtaBank and associated tools are different from other database systems currently in use, as described further below.

Example embodiments will be described more fully hereinafter, in which example embodiments are described. It should be understood that such systems, computer readable media, and methods may be embodied in many different forms and should not be construed as limited to the example embodiments set forth herein. Rather, these example embodiments are provided so that this disclosure will be thorough and complete, and will fully convey the scope of the claims to those of ordinary skill in the art.

The term “machine learning” as used herein generally refers to a type of AI that provides computers with the ability to learn without being explicitly programmed. Machine learning is a branch of AI focusing on systems that can learn from data, identify patterns, and make decisions with minimal human intervention.

As used herein, the term “full length native protein” refers to a protein that is in its native or natural state and unaltered by any denaturing agent such as heat, chemical mutation or enzymatic reactions. A wild-type protein would be considered a full length native protein. The term full length native protein sequence, as used herein, refers to the amino acid sequence found in the full length native protein.

As used herein, “mutation” refers to a change in the amino acid sequence of a native protein. Mutations can be described by using the native sequence and then identifying the specific acid that have been changed. A “mutant” refers to the protein that contains the mutation. A full length mutant sequence refers to the full amino acid sequence of the mutant protein, instead of describing the mutant as the amino acids that are different from the native protein.

Terms such as “first”, “second”, and “within” are used merely to distinguish one component (or part of a component or state of a component) from another. Such terms are not meant to denote a preference or a particular orientation, and are not meant to limit embodiments of the disclosure. In the following detailed description of the example embodiments, numerous specific details are set forth in order to provide a more thorough understanding of the invention. However, it will be apparent to one of ordinary skill in the art that embodiments of the disclosure may be practiced without these specific details. In other instances, well-known features have not been described in detail to avoid unnecessarily complicating the description.

A user may be any person or entity that interacts with the database, the AI platform, or both. Examples of a user may include, but are not limited to, a principal investigator, a scientist, a post-doctoral candidate, a graduate student, or a pharmaceutical company, for example. There can be one or multiple users.

This disclosure describes an embodiment of a database and associated tools, herein referred to as ProtaBank, for storing and searching all types of PE data, spanning a wide range of properties, including those related to activity, binding, stability, folding, and solubility. The database organizes, integrates, annotates and structures mutational data obtained from diverse approaches, including computational and other types of rational design, saturation mutagenesis, directed evolution, and deep mutational scanning. ProtaBank's functionality permits accurate comparisons between different data sets which facilitates sharing PE data with collaborators and improves the usability of PE datasets for data mining and other analysis methods. ProtaBank's analysis tools help users gain insights into sequence-activity and structure-activity relationships, improve understanding of how proteins function, and leads to the design of proteins with new and improved properties.

Unlike other protein databases, such as PDB, Gen Bank, ProTherm, Uni Prot, and BRENDA, ProtaBank can be designed for all types of PE data spanning a wide range of properties and can deposit data from a variety of sources into storage in forms that are easily accessible and analyzable. ProtaBank can also store the entire protein sequence for each of the variants instead of just the mutations and offers detailed descriptions of the experimental assays used. These features are incorporated to allow for accurate comparisons of measurements across multiple studies or groups, making it easier to identify trends and determine how different assays, parameters, or conditions affect the results.

Also disclosed is an artificial intelligence (AI) platform, which serves to connect the fields of protein engineering and machine learning. As described above, the ProtaBank database is a central repository to store, organize, and share protein mutation data spanning a broad range of properties. The ProtaBank AI Platform fully realizes the potential of this database by bringing together dataset creation and preparation tools, protein encodings, and ML protocols to enable machine-directed protein engineering based on the data in ProtaBank. The flexible design of the platform coupled with interfaces to popular ML frameworks allows scientists to discover new predictive algorithms and build models tailored to their data and problems.

The background of the disclosure describes current limitations in protein engineering. The current disclosure uses ProtaBank with one or more of AI and ML methods to overcome these limitations. In some embodiments, the ProtaBank AI Platform comprises AI and ML approaches which learn how to correlate protein function with sequence mutations where a score function is not needed to generate results as ProtaBank can comprise a large amount of data for accurate training. Recent advances in lab automation and deep sequencing have made generating and collecting this data much easier and more routine, and ProtaBank's data collection algorithms are able to parse this data efficiently. 3D protein structures are also not needed within the ProtaBank AI Platform, but in some embodiments are used. In embodiments of the disclosure, AI methods correlate measured data with sequence variation, structural information, while in some embodiments they do not. An understanding of how the protein functions is similarly unnecessary, and unlike DE, the ProtaBank AI Platform methods can take advantage of data over large regions of sequence space. In embodiments, the ProtaBank AI Platform predicts sequences that simultaneously optimize multiple protein properties, for instance fitness and solubility. That is, the ProtaBank AI Platform can predict protein function from sequence mutation data. The AI Platform can be comprised on computer executable instructions for execution by a processor. The computer executable instructions with the AI Platform can be stored in a storage repository, such as a hard drive or memory, for example.

In embodiments, the AI platform comprises guided protein engineering using large amount of data. In embodiments, large amounts of data is greater than hundreds of data points to more than a million data points. A specific embodiment could have greater than a thousand data points. In embodiments, ProtaBank comprises data including specific properties like binding to a target including large protein variant-drug target binding datasets (e.g., data collected via deep sequencing and screening or selection of combinatorial protein variant libraries). In embodiments, the AI platform can create initial ML models which can be trained to predict improved sequence variants. These improved sequence variants can then be assayed experimentally, and the results used to repeat the process. Embodiments of the disclosure include facilitating protein design and experimental procedure workflow by providing tools that make the process from generating experimental datasets to predicting new beneficial protein variants as seamless and easy as possible. In embodiments, additional properties like expression and serum half-life are used to create broadly predictive learned models using datasets from a range of different proteins. For example, predicting the viscosity of an antibody-based protein therapeutic may not require data for the specific antibody of interest, but instead a more general model may be trained on existing antibody viscosity measurements from earlier antibody engineering projects. An embodiment of the disclosure is a central repository designed to collect and organize protein sequence mutation data for many different properties and to facilitate the creation of ML datasets from the accumulated data is a necessary component for AI protein engineering. The ProtaBank database and associated ProtaBank AI platform is one example of such an embodiment; however, it should be understood that the description below of the specific implementation of the ProtaBank database, associated tools, and AI platform are not limiting to the disclosure, but are instead specific example embodiments.

Example Database Construction and Content

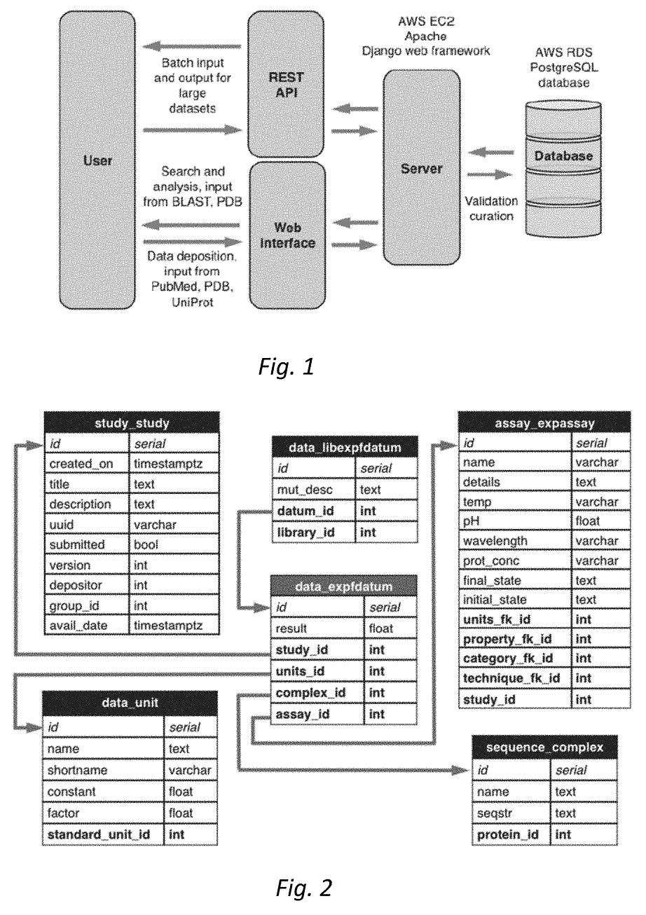

In embodiments, the protein mutational database is described and implemented as ProtaBank. ProtaBank comprises three main functionalities: (1) data deposition tools (2) data storage, and (3) tools for data searching and analysis. An example design and workflow for ProtaBank is summarized in FIG. 1. As shown in FIG. 1, users can interact with ProtaBank through the web interface or the REST API. Data sent to the server is validated and curated before final submission into the database. In embodiments, ProtaBank comprises a central repository for storing and sharing the world's published protein sequence mutation data, in much the same way that GenBank is a central repository for nucleic acid sequences. In some embodiments, ProtaBank collects sequence mutation data for a wide range of properties that address all aspects of protein engineering in drug discovery and development including, for example, stability, expression, binding, activity, solubility, aggregation, and half-life, viscosity, immunogenicity, crystallizability, spectral properties, toxicity, bacterial resistance, and specificity. In some embodiments, ProtaBank employs standard formats that facilitate comparison of results across different datasets. Example, standard formats include standardized assay conditions for stability measurements including temperature, concentration, and pH. In some embodiments, ProtaBank comprises search and analysis tools and collection utilities. In additional embodiments, the search and analysis tools and collection utilities are used to create datasets for machine learning. In some embodiments, ProtaBank integrates a company's proprietary protein sequence mutation data with the ProtaBank public data while securely protecting the proprietary protein sequence mutation data. In some embodiments, ProtaBank provides an organization-wide centralized repository to track, persist, and maintain a company's valuable protein sequence mutation data for later use in AI and other traditional protein engineering projects.

Systems of the disclosure can include an intranet-based computer system that is capable of communicating with various software. A computer system includes any type of computing device and/or communication device. Examples of such a system can include, but are not limited to, super computers, a processor array, distributed parallel system, a desktop computer with LAN, WAN, Internet or intranet access, a laptop computer with LAN, WAN, Internet or intranet access, a smart phone, a server, a server farm, an android device (or equivalent), a tablet, smartphones, and a personal digital assistant (PDA). Further, as discussed above, such a system can have corresponding software (e.g., user software, sensor device software). The software of one system can be a part of, or operate separately but in conjunction with, the software of another system.

Embodiments of the disclosure include a storage repository. The storage repository can be a persistent storage device (or set of devices) that stores software and data. Examples of a storage repository can include, but are not limited to, a hard drive, flash memory, some other form of solid state data storage, or any suitable combination thereof. The storage repository can be located on multiple physical machines, each storing all or a portion of the database, AI platform, protocols, algorithms, and/or other stored data according to some example embodiments. Each storage unit or device can be physically located in the same or in a different geographic location. In embodiments, the storage repository may be stored locally, or on cloud based serveries such as Amazon Web Services.

In one or more example embodiments, the storage repository stores one or more databases, AI Platforms, protocols, algorithms, and stored data. The protocols can include any of a number of communication protocols that are used to send and/or receive data between the processor, datastore, memory and the user. A protocol can be used for wired and/or wireless communication. Examples of a protocols can include, but are not limited to, Modbus, profibus, Ethernet, and fiberoptic.

Systems of the disclosure can include a hardware processor. The processor of the executes software, algorithms, and firmware in accordance with one or more example embodiments. The processor can be a central processing unit, a multi-core processing chip, SoC, a multi-chip module including multiple multi-core processing chips, or other hardware processor in one or more example embodiments. The processor is known by other names, including but not limited to a computer processor, a microprocessor, and a multi-core processor. The processor can also be an array of processors.

In one or more example embodiments, the processor executes software instructions stored in memory. Such software instructions can include generating machine learning models, executing machine learning models, performing analysis on data received from the database, and so forth. The memory includes one or more cache memories, main memory, and/or any other suitable type of memory. The memory can include volatile and/or non-volatile memory.

The processing system can be in communication with a computerized data storage system which can be stored in the storage repository. The data storage system can include a non-relational or relational data store, such as a MySQL. or other relational database. Other physical and logical database types could be used. The data store may be a database server, such as Microsoft SQL Server., Oracle., IBM DB2., SQLITE., or any other database software, relational or otherwise. The data store may store the information identifying syntactical tags and any information required to operate on syntactical tags. In some embodiments, the processing system may use object-oriented programming and may store data in objects. In these embodiments, the processing system may use an object-relational mapper (ORM) to store the data objects in a relational database. The systems and methods described herein can be implemented using any number of physical data models. In one example embodiment, an RDBMS can be used. In those embodiments, tables in the RDBMS can include columns that represent coordinates. The tables can have pre-defined relationships between them. The tables can also have adjuncts associated with the coordinates.

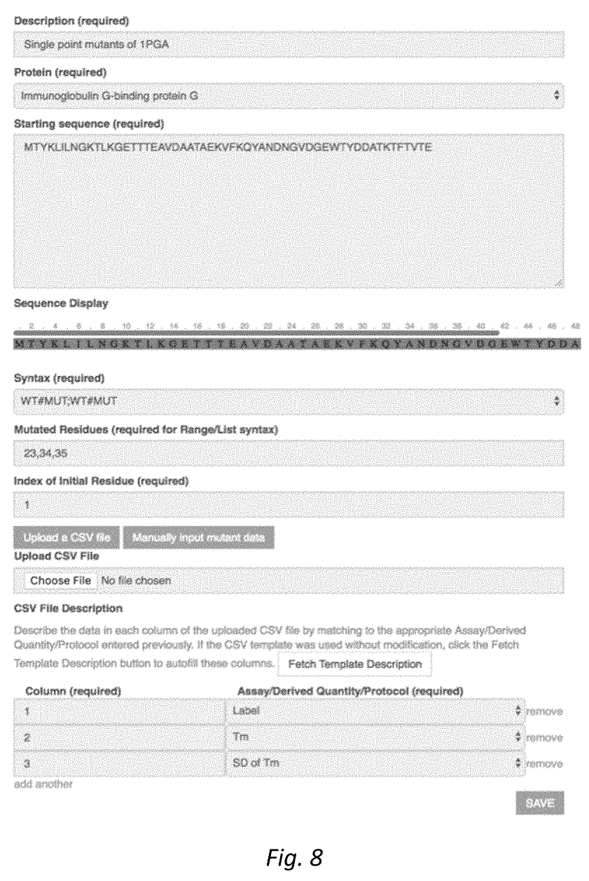

In embodiments, the systems of the disclosure can include one or more I/O (input/output) devices allow a user to enter commands and information into the system, and also allow information to be presented to the user and/or other components or devices. Examples of input devices include, but are not limited to, a keyboard, a cursor control device (e.g., a mouse), a microphone, a touchscreen, and a scanner. Examples of output devices include, but are not limited to, a display device (e.g., a display, a monitor, or projector), speakers, outputs to a lighting network (e.g., DMX card), a printer, and a network card. For example, the input devices can be used to enter data on native proteins and mutation sequences and assays (i.e. FIG. 8). The input devices can also enter wanted functional data for a protein. The output devices can be used to output analysis data and/or engineered protein sequences resulting from AI protein design.

FIG. 11 is a non-limiting example system for engineering proteins 1100. It comprises a computer 1102, a processor 1104, a memory 1106, and a storage repository 1108. The storage repository 1108 can comprise a database 1110. Input/Output devices 1112 are connected to the computer 1102 and usable by a user. A bus (not shown) can allow the various components and devices to communicate with one another. A bus can be one or more of any of several types of bus structures, including a memory bus or memory controller, a peripheral bus, an accelerated graphics port, and a processor or local bus using any of a variety of bus architectures. A bus can include wired and/or wireless buses. The components shown in FIG. 11 are not exhaustive, and in some embodiments, one or more of the components shown in FIG. 11 may not be included in a specific embodiment. Further, one or more components shown in FIG. 11 can be rearranged. Is should also be understood that in embodiments, the various elements shown here can be located together or located remotely from each other. For example, the database could be stored in a different location, such as on a server, from the processor used by the AI Platform.

Various techniques are described herein in the general context of software. Generally, software includes routines, programs, objects, components, data structures, and so forth that perform particular tasks or implement particular abstract data types. An implementation of these modules and techniques can be stored on or transmitted across some form of computer readable media. Computer readable media is any available non-transitory medium or non-transitory media that is accessible by a computing device. By way of example, and not limitation, computer readable media includes computer storage media.

Data Deposition and Curation

Protein engineering data is diverse and complex, and describing and depositing the data creates a burden on the individual user. In embodiments, ProtaBank solves this problem by a suite of data deposition tools. In this example, ProtaBank's data deposition tools are designed to accept the wide range of data generated in PE efforts and to automate the process so as to facilitate entry and ensure accuracy. In embodiments, publication details (e.g., authors, title, journal, date, abstract, for example) can be fetched from PubMed, and the protein sequence can be retrieved from the PDB or UniProt. If available, structural data for the protein can be fetched from the PDB. In embodiments, two modes of data deposition are provided: an interactive web interface that supports upload of data in a spreadsheet format (i.e., via a comma-separated values (CSV) file, Excel spreadsheets, for example) (FIG. 8), and a REST API layer that allows for programmatic batch upload of data.

FIG. 8 is a screenshot of the ProtaBank data deposition form for data in a mutant library. A short description of the data set, the protein that was mutated, and the starting sequence are specified, along with the format (syntax) used to describe the mutants. The mutational data can be uploaded from a CSV file or entered manually. This screenshot shows the form for a CSV file upload: the data in each column of the CSV file is specified by selecting the appropriate Assay/Derived Quantity/Protocol for the property from the drop-down menu.

In embodiments, ProtaBank's web interface specifies a description of the methods used to assay the protein mutants. For example, for each assay, the deposition tool may collect an assay name, the category of protein property that was engineered or studied, the specific property measured, the technique employed, and the units used. Additionally assay conditions such as temperature, concentration, pH, binding partners could be used. Also included could be uncertainties such as standard deviation/error and mathematical formulas indicating how a property was calculated. In embodiments, items except the assay name can be specified by selecting from options in a drop-down menu. By entering this information, assays can be clearly defined and compared.

For example, PE data can be input in two forms: as individual mutants or as a mutant library (a set of mutant sequences obtained by mutating a specified set of residues in a protein). Mutational data can be uploaded from a CSV file or it can be entered manually on the web form, for example. The data entry page for a mutant library is shown in FIG. 8. To specify a mutant library, the user can first enter the starting protein sequence from which the mutations were made. All mutants in the library can then be described either by their full sequence or as mutations from this starting sequence. Two example formats for the latter are: (1) the WT#MUT format (e.g., M3A+V5L+S19T) and (2) the mutated residue range/list format in which a range or list of residues is specified that correlates positions in the starting sequence with the amino acids given in the CSV file (e.g., QRS for residues 3-5; or QRS for residues 3,5,7). ProtaBank can then take the description of mutants entered, parse the data, and store it as full amino acid sequences. This method makes it possible to validate the accuracy of mutant data provided in the WT#MUT (wild-type amino acid, residue #, mutant amino acid) format; i.e., the wild-type amino acid listed for each of the mutated positions is compared to what is specified in the starting sequence, and any discrepancies can be flagged.

In embodiments, submitted data are validated to ensure data integrity before inclusion in the database. Automated tests can be performed to ensure that: (1) the data falls within the correct range of values (e.g., temperature in K must be a non-negative number), (2) the assigned units are appropriate for the assayed property, and/or (3) the amino acid listed for wild type is consistent with that specified in the starting sequence (for mutants described in the WT#MUT format). Outliers in a data set are also flagged and the submitter is asked to check for accuracy. Policies can be implement that handle data that fail validity testing.

### Example Embodiment of Database Schema

In an embodiment, ProtaBank is implemented as a relational database using the PostgreSQL database. In this embodiment, the highest level of organization is a study corresponding to a PE effort. Each study can have four core tables to describe the PE data: sequence_complex, assay_expassay, data_expfdatum, and data_units which respectively represent the sequence of a given protein mutant, the experimental assay that was used to probe the property of interest, the numerical value obtained for the mutant (i.e., the assay results), and the units associated with the numerical value (FIG. 2) FIG. 2 shows the table for experimental data represented by a number (data_expfdatum) and all tables with foreign key relationships to it. Each table shows the field name (left) and the variable type for the field (right). Each datum in the data_expfdatum table has a foreign key relationship (arrows) to a study table (study_study) that organizes the context in which the experiments were performed, an assay table (assay_expassay) that describes the procedure used to obtain the measurement, a sequence table (sequence_complex) that holds the protein sequence of the mutant, and a units table (data_unit) that describes the units of the result. For data that is part of a mutant library, a foreign key links it to the library table (data_libexpfdatum). Analogous tables exist for data obtained from computations/simulations, derived data, and qualitative or range data. The native sequence can be defined in a data_library table (not shown in the figure) which collects information relating to a related subset of study data with a common native sequence, mutated residues, syntax, and other properties. A study may have multiple libraries.

The embodiment shown in FIG. 2 of ProtaBank has separate corresponding tables to represent computational protocols and derived quantities, and to store qualitative data (e.g., folded/unfolded) or data expressed in terms of a range or limit (e.g., 20-30, >100). In addition to the core data tables, each study can include publication information, structural data on the protein that was engineered (i.e., the PDB file, if available), and experimental gene construct information. This type of information adds context and additional query and filter parameters to the PE data. Non-published PE studies can also be input in a similar fashion. In these cases, the researchers and organizations involved are specified instead of the authors and affiliations. In embodiments, ProtaBank can be structured so that depositors of non-published results may embargo the release of the data until publication.

In embodiments, the ProtaBank schema design incorporates two main elements: (1) the full amino acid sequence of the protein is stored to facilitate comparison of mutants across different assays and studies, and (2) for each assay, information about the protein property measured, the assay conditions and techniques used, and the units of the resulting data is collected in addition to the results. Although these requirements necessitate the application of special methods and procedures for deposition and curation, they are included in embodiments for the following reasons.

First, PE studies and databases typically describe a mutant by listing the changes to its protein sequence relative to a specified starting sequence. However, the starting sequences used in engineering a given protein are often not the same across studies, which can cause confusion and makes comparisons challenging. The wild-type protein is not always used; residues may be changed, added, or deleted at the termini, for example, to facilitate expression or purification, or substitutions may be made to make the protein more amenable to the assay conditions. Many mutant databases only store the mutational data for the positions mutated. For example, M3A+V5L+S19T might be used to identify a mutant that has been mutated to Ala, Leu, and Thr at positions 3, 5, and 19, respectively; the rest of the sequence (the background in which the mutations were made) is either not given or not recorded. Not knowing the entire sequence for each mutant confounds comparisons, as any differences in the reported results could be due to differences in the background residues.

Second, comparison across studies may be difficult due to differences in assay conditions or techniques, which can greatly affect the results. Embodiments of the ProtaBank schema takes these issues into account. As outlined above, the database uses the assay_expassay table to describe the procedure that was used to determine a given protein property. This table has foreign key relationships with a series of other tables (category, property, technique, units) that help categorize and describe the many ways these properties can be measured. The category table provides the general type of protein property that was engineered or studied (e.g., stability, activity, binding). The property table is more specific and describes the property that was actually measured and gave rise to the result [e.g., melting temperature (Tm), catalytic rate constant (kcat), dissociation constant (Kd)]. A non-exclusive list of example categories and properties which can be included in ProtaBank are found in Table 1. Commonly used experimental or computational techniques can also be provided to indicate how the property was assayed (e.g., circular dichroism, surface plasmon resonance, for example). Note that the properties and techniques supplied are not comprehensive, and users can enter additional ones. Finally, the units table contains commonly used units that are appropriate to the property measured. For example, the units available for the Gibbs free energy of folding/unfolding (ΔG) are kcal/mol and kJ/mol. This level of description is designed to provide enough detail so that data collected from different sources can be compared and analyzed appropriately.

TABLE 1
Example protein properties included in ProtaBank

| Category | Properties |
|---|---|
| Activity | Activity, Catalytic efficiency (k_{cat}/Kₘ), Catalytic rate constant (k_{cat}), |
| | Count/Number, EC50, Energy, Enrichment, Epistasis, Fitness, IC50, |
| | Inhibition constant (Kᵢ), Maximal rate (Vₘₐₓ), Michaelis constant (Kₘ), |
| | Relative activity, Specific activity |
| Binding | Association constant (Kₐ), Binding affinity, Count/Number, |
| | Dissociation constant (K_{d}), ELISA, Energy, Enrichment, Enthalpy of |
| | binding (ΔH), Entropy of binding (ΔS), Epistasis, Fitness, Frequency |
| | of occurrence, Gibbs free energy of binding (ΔG), Inhibition constant |
| | (Kᵢ), Rate constant of association (kₒₙ), Rate constant of dissociation |
| | (k_{off}) |
| Expression | Concentration, Energy, Optical density (OD), Enrichment, Frequency |
| | of occurrence, Minimum inhibitory concentration (MIC), Yield |
| Growth | Antimicrobial resistance, Energy, Enrichment, Frequency of |
| | occurrence, Optical density (OD) |
| Preclinical/ | Bioavailability, EC50, Half-life (t_{1/2}), IC50, Immunogenicity, Toxicity |
| Clinical | |
| Solubility/ | Concentration, Energy, Fractional increase in solubility, Insoluble |
| Aggregation | fraction, Oligomerization state, Soluble fraction |
| Specificity | Energy, Frequency of occurrence, Relative activity, Relative affinity, |
| | Relative k_{cat}, Relative k_{cat}/Kₘ, Relative K_{d} |
| Spectral | Brightness, Emission wavelength (λₑₘ), Energy, Excitation wavelength |
| properties | (λₑₓ), Extinction coefficient, Fluorescence intensity, Maturation half- |
| | time, Photobleaching half-time, pKa, Quantum yield |
| Stability/ | Constant pressure heat capacity of unfolding (ΔCₚ), Count/Number, |
| Folding | Denaturant concentration at midpoint of unfolding transition (Cₘ), |
| | Energy, Enthalpy of unfolding (ΔH), Entropy of unfolding (ΔS), |
| | Equilibrium constant (K), Gibbs free energy of folding/unfolding (ΔG), |
| | Melting temperature (Tₘ), Rate of folding (k_{F}), Rate of unfolding (k_{U}), |
| | Slope of chevron plot (m), Slope of the denaturant unfolding |
| | curve/cooperativity value (m), Temperature of maximum stability, |
| | Thermal tolerance, ß-Tanford value, Φ-value |

Note:
Additional categories and/or properties can be specified by the user. Standard deviation or standard error data can be saved for any property.

Embodiments of the disclosure include added features that allow users to input and save mutant data for each of the chains in multi-chain proteins (e.g., antibodies) and/or to specify and search on the binding partner in complexes with other proteins, ligands, or small molecules (e.g., in binding studies or enzyme-substrate interactions).

Data Deposition Tools

Embodiments of the disclosure include partially or fully automating the process of identifying publications with relevant protein mutation data. Articles with relevant protein mutation data can be identified based on the title, abstract, and keywords. These tools can automatically identify and extract protein IDs and sequences, mutational data, and author names and contact information from article text, figures, and supplementary information. Once extracted this information can be compiled and used to assemble an article summary.

Some embodiments of ProtaBank include a step-by-step study entry page, improving data entry for studies with multiple proteins, multiple chains, or multiple-component-complexes (such as enzyme-substrate, antibody-antigen, protein-ligand), supporting SMILES (simplified molecular-input line-entry system) string inputs, and a multiple sequence viewer can be added to assist users during the data input process. Additional embodiments of the disclosure are a graphical tool for specifying residue numbering, support for commonly used biological file formats such as fasta and fastq, tools for entry of equations and special characters, multiowner permissions for users to easily modify publication details or make revisions, and commonly used data reporting formats used in the literature or by the users.

In some embodiments, ProtaBank includes integrations between ProtaBank and the Research Collaboratory for Structural Bioinformatics (RCSB) PDB (the regional center in the USA) to allow users to easily navigate between structure data and mutation data for a particular protein or family of proteins. In this way users can seamlessly explore protein structure, sequence, and mutation space across these major databases. In some embodiments, standardized formats are used for the description of protein sequence, structure, and properties.

### Example Search, Analysis, and Other Tools

An embodiment of ProtaBank offers several search and analysis tools that allow users to: (1) browse and search for relevant studies queried by publication/study details (title, abstract, author), protein name, PDB ID, UniProt accession number, or protein sequence, (2) identify data and mutants related to a given protein sequence by BLAST search, (3) visualize mutational data mapped onto a three-dimensional (3D) protein structure, and/or (4) compare and correlate data measured using different assays. Example 2 of the disclosure illustrates the use of example tools. Embodiments of the disclosure include analysis tools which find statistical correlations between various data elements. In embodiments, users can identify data by keyword search of the study title, abstract, protein name, publication author or date, PDB ID, and UniProt ID. Data from across studies can be queried using a BLAST sequence search to identify relevant sequences, assays, and protein properties.

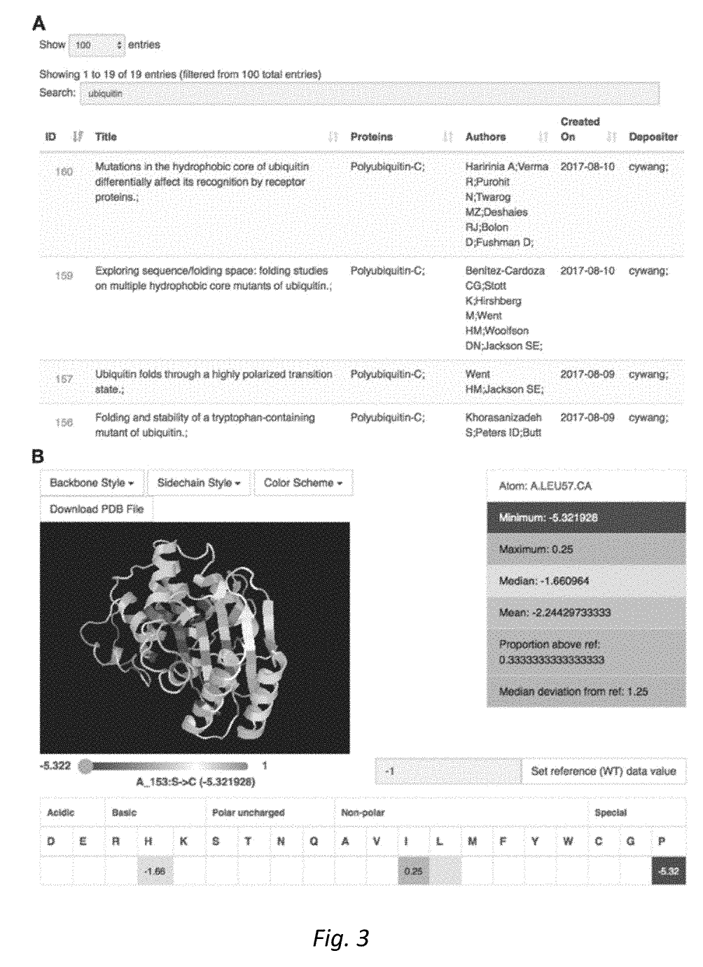

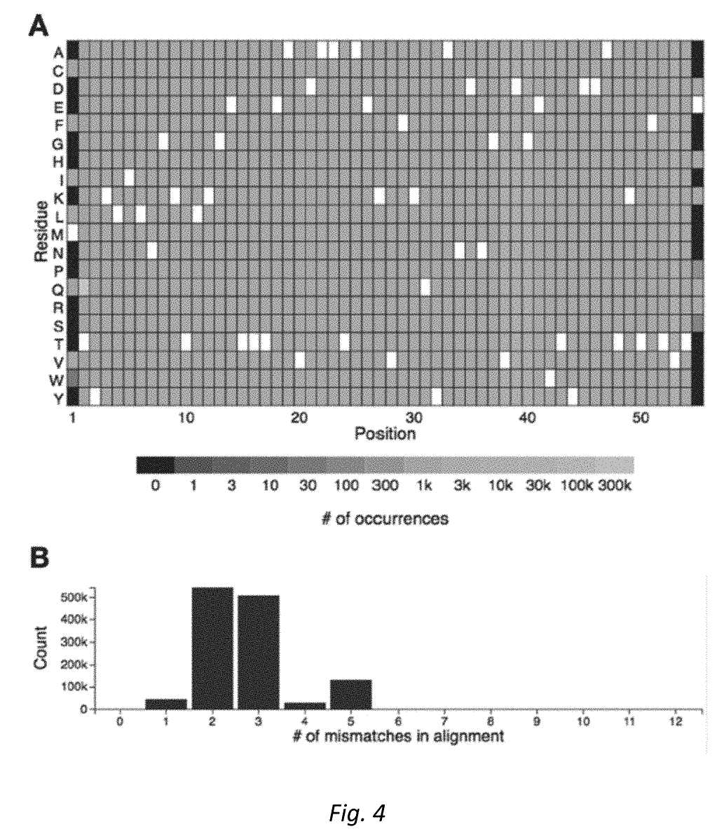

Embodiments of the disclosure include analysis tools for comparison and/or summarization of the data within ProtaBank. For individual studies, the distribution of the data can be visualized and relevant statistics calculated. One can identify closely related mutants to a given sequence, and distributions of the number of mutations away and the amino acids involved in those mutations can be obtained (FIGS. 3 and 4). For a set of related sequences, and data can be ranked and sorted by property.

Some embodiments include a companion version of the ProtaBank database as a micro service within a company's own technology stack. This addresses any privacy concerns for proprietary data. These companion databases can be automatically updated when any new data is approved for addition to ProtaBank. A companion database can include customer-specific proprietary protein engineering data, for example.

Embodiments of the disclosure include a database with an added embargo feature so that unpublished data can be kept private until a specified date (for example, up to 6 months away) and users need not wait until after publication to enter data into ProtaBank.

### Example ProtaBank AI Platform

This platform integrates: (1) protein sequence mutation data from the public and proprietary ProtaBank databases, (2) AI framework, (3) encoded sequence and structure features using protein domain knowledge for enhanced ML, and (4) a workflow that makes powerful AI drug development approaches highly accessible. In embodiments, the AI Platform predicts new protein sequence variants to be validated experimentally. In some embodiments, the AI Platform provides all the tools needed for AI drug development in a single platform. For example, these tools can be data encodings (including protein specific data encodings based on evolutionary data, protein structure, and computed features/predictions from protein analysis and CPD algorithms), data normalization and regularization, synthetic data generators, and dataset tools such as data selection, data filtering, data cleaning, data harmonization, data sampling. In some embodiments, the AI Platform can optimize several protein properties simultaneously. In some embodiments, the AI Platform includes the 3D protein structure. In some embodiments, the AI Platform does not use a 3D protein structure.

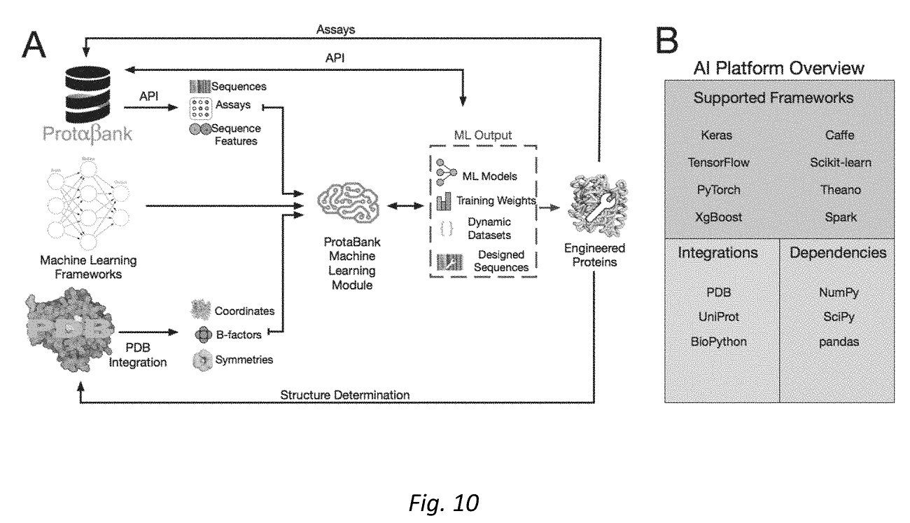

In one embodiment, output of the platform and included tools is available in Python based tensor formats as described in FIG. 10B. Any AI Framework which accepts tensor data in one of these formats can be supported. AI Frameworks which accept data in a similar format may also be supported with the proper data translation utility. For example, frameworks can include Keras, Caffe, TensorFlow, Scikit-learn, PyTorch, Theano, XgBoost, and Spark.

In embodiments, the AI Platform and ProtaBank database represents a full encapsulation of the machine-guided protein engineering process outlined in FIG. 10, helping researchers identify promising candidates for experimental screening. Embodiments of this platform brings together ProtaBank data with novel protein encodings learned from many years of CPD, structural and evolutionary data from third party databases, validated protein normalization strategies, and/or the cutting edge published ML protocols for protein engineering. These additions enable the concurrent engineering of multiple properties, predictors applicable to more generalizable classes of proteins, and generative models that explore isolated areas of the protein functional landscape. Progress in any of these areas would represent a significant breakthrough in protein engineering.

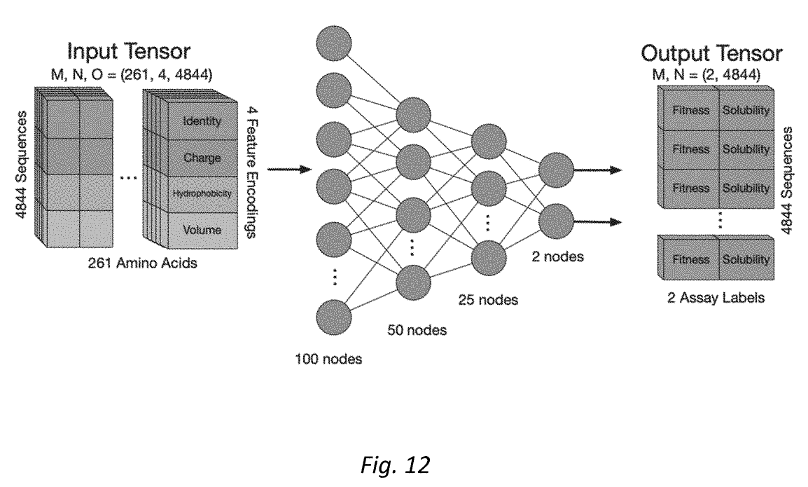

FIGS. 10A and 10B are illustrations of layouts for an example AI Platform integrated with the ProtaBank database. FIG. 10A illustrates how the ProtaBank AI Platform integrates input data from the ProtaBank mutation database (upper left), which provides information such as sequences, assays, and sequence features through an API, and the PDB structure database (lower left), which provides information such as coordinates, B-factors, and symmetries, with the use of a chosen ML framework (middle left) into a ProtaBank Machine Learning Module (PMLM) to encode, normalize and featurize the data into numerical tensors/arrays. These can be processed by any Python machine learning toolkit to create and train models as is shown in FIG. 12. Model predictions guide the creation of newly engineered proteins. This platform can be used in an iterative process, where measured assay data from the newly engineered proteins is deposited back into ProtaBank and used to retrain ML models. Structure information can also be deposited into the PDB to be used in the next protein engineering project. FIG. 10B illustrates examples of how the AI Platform can support several ML frameworks to provide algorithm flexibility using the NumPy, SciPy, and pandas data formats as a common language. Integrations with the PDB and UniProt can make structural and sequence data available to the machine learning module. An integration with BioPython can provide a common set of tools for customizing platform tools.

In some embodiments, the AI Platform includes an AI training module. Machine learning models may be generated by the AI platform during an initial set-up process or after receiving instructions provided by a user. For example, the AI Platform could generate a machine learning model by training on a subset of proteins found in ProtaBank. Example of training during set up could include training on a subset of a specific type of protein, for example, antibodies, membrane bound proteins, metalloproteins, tyrosine kinases, proteases, globular proteins, and beta-barrel proteins, to generate an AI machine learning model for each type or subset of protein.

In another embodiment, the AI Platform could generate a machine learning model based on a user specified protein of interest. In this example, the ProtaBank database would find close matches to the protein of interest, for example, proteins with similar functions or with a certain percent similarity to the protein of interest (20%-99% similarity, for example), then take a subset of the proteins identified in the database to use as a training set. The percent similarity, for example, could be a user supplied value.

AI training modules and AI machine learning models can include any of the data from the ProtaBank database, include full length native protein sequences, full length mutant protein sequences, differences between the sequences, data associated with full length native sequences and data associated with a full length protein sequences, any of the protein properties found in Table 1, and assay data. The training modules can be trained to optimize protein sequence in order to effect functional properties of the protein, for example, any of the characteristics found in Table 1, including efficacy, binding affinity, and serum half-life. Proteins can also be engineered for proper function or activity, such as binding to a drug target or deactivation of disease-causing biomolecules, other properties like expression level, solubility, and serum half-life can also be maintained or improved.

Embodiments of the disclosure include collecting fully described and structured data on a range of protein properties enabling multi-property dataset generation. Multi-task machine learning models can then consider all of the relevant data when making predictions. In some embodiments, the AI Platform comprises multi-task neural networks. ProtaBank and the ProtaBank AI Platform provide the data and methods for developing and validating multi-task models over a range of protein engineering applications. Further, the AI Platform can include dynamic design protocols that query and apply ProtaBank data to improve CPD results.

Embodiments of the AI Platform can include iterative data. For example, given the specific function wanted from the protein the AI platform can design one or potential protein sequences whose proteins would fit the wanted function. The sequences could then be made into proteins, assayed for characteristics and/or function, and then the data from the assays could be reentered into the database as mutational data, further refining designs predicted by the AI platform. Function when referred to in a CPD context, can refer to the characteristics listed in table 1.

In embodiments, the AI Platform comprises a machine learning method, such as a neural network for effective protein function prediction. In some embodiments, the AI platform includes neural networks, genetic algorithms, decision trees, fuzzy logic, symbolic rules, gradient boosting, support vector machines, and other machine learning based systems. Pluralities and/or combinations of the above may also be used. In embodiments, the AI Platform can use ML frameworks such as, Keras, Caffe, Pytorch, TensorFlow, the Microsoft Cognitive Toolkit, MXNet, Chainer, and Theano, with a Python implementation as the predominant data science language. In embodiments, the AI platform will allow for agnostic integration with other algorithms (such as gradient boosting, SVM, Gaussian processes) and their respective frameworks (XGBoost, SciKit Learn, GPy etc.) by separating data preparation from model creation and by using a NumPy data format common to all of these frameworks. In some embodiments, data preparation tools can be released as a Python package.

Embodiments of the disclosure use protein feature encodings to add physical or biological knowledge to amino acid sequences to create representations amenable to machine learning. As the choice of encoding varies based on the size and diversity of the input, as well as the task, several encoding methods can be implemented, allowing users to test and select the encodings most relevant to their problem. The AI Platform can include the following encodings, for example: one-hot, autoencoders, amino acid property encoders, learned BLOSUM/MSA evolutionary encodings, sequence mutation representation relative to WT, secondary structure/solvent accessible surface area encodings, learned AA embeddings, POOL, Phoenix, and/or structural/graph/topological encodings.

In some embodiments, the AI platform generates an input tensor from mutant protein sequences in a training set. The input tensor is created by encoding features generated from the mutant sequences. In some embodiments the input tensor is generated by encoding features generated from a combination of the mutant protein sequence and a computer generated model of the mutant three dimensional protein structure. Other embodiments include generating input tensors from different combinations of encoded features. Features may include but are not limited to the identity, charge, hydrophobicity, probability of amino acid type preceding or following each amino acid, net charge of the sequence for a window of various amino acid length centered on an amino acid and/or volume for each amino acid in the sequence (FIG. 12A). In embodiments, an output tensor is generated comprising characteristic data of the mutant sequences used to generate the input tensor. This input tensor along with its corresponding output tensor is used to train a predictive machine learning model. The machine learning model can output predictions for novel mutant protein sequences comprising fitness and/or protein functional data, such as the protein functional data found in Table 1 (FIG. 12A). These predicted characteristics are used in a selection process composed of a ranking algorithm to order the corresponding mutant sequences based on desired protein characteristics. Rankings can be accomplished through comparing the predicted characteristics to wanted characteristics and taking the closest matching 10, 20, 30, 40, 50, 60, 70, 80, or 100, for example. The rankings can also be done by taking the synthetic mutant sequences which have predicted characteristic that are within a certain percentage of the wanted characteristic, for example.

In embodiments, prior to AI learning, protein-specific data is normalized and/or regularized. In some embodiments, ProtaBank comprises additional tools to address the distribution of reasonable values for a particular protein property, reported measurement error, and outlier handling. This can include assay magnitude normalizers and encoding normalizers.

In embodiments, the output of the AI Platform is one or more synthetic protein sequences. In some embodiments, the AI Platform includes sequence mutators based on random and non-random distributions, as well as support for generative networks. A user can then synthesize the synthetic protein sequences and test for function.

In embodiments, a machine learning model is trained using encoded protein features and normalized assay labels to create a predictor of assay labels given a mutated protein sequence.

In embodiments, the output of the AI Platform is one or more synthetic protein sequences. In some embodiments, the AI Platform includes methods for generating trial protein sequences, for example: sequence mutators based on random and non-random distributions, and generative networks. These trial sequences can then be assessed using the trained machine learning predictor and selected for sequence diversity and predicted function. A user can then synthesize these trial sequences and verify function experimentally. Newly generated data can then be deposited back in ProtaBank to generate a new, improved set of trial sequences for the next round of testing.

In some embodiments, model topologies and parameters will be selected and tested on a per-dataset and per-encoding basis. For instance, multi-task neural networks can be used for datasets studying multiple protein properties, while convolutional neural networks can be tested for topological or spatial encodings.

In embodiments, users can download the AI Platform and incorporate it into their machine learning workflow to guide experimental drug discovery. These tools can integrate seamlessly to access the public data in ProtaBank or proprietary data in the secure companion database.

In some embodiments, dataset creation tools are used to generate context-specific data subsets (e.g., only stability, expression, and solubility assays for a group of related proteins). As described above, rigorous collection and structured storage of experimental assay techniques, properties measured, conditions, and other metadata enables the identification and analysis of relevant data. ProtaBank's query APIs can be used to support the creation of ML-friendly curated datasets and develop a set of tools within the ProtaBank AI Platform to assist in preparing and combining data from several studies into a single dataset. In embodiments of the disclosure, Protabank comprises tools to enable the following: data selection based on sequence/structural identity, protein property, and assay condition; data filtering to exclude proline mutations, fold changes, membrane proteins, data with high standard error, and other conditions; data cleaning to transform missing, range based, or categorical data; data harmonization to combine data from multiple studies; automatic tools for unit conversion and sequence/mutation mapping; tools to quantify study/assay overlap and correlation to help users select studies and develop customized harmonization functions; and/or data sampling to create even, non-redundant, and distinct training and test sets for ML.

In some embodiments, the AI Platform includes dynamic design protocols. Specific embodiments can include a dynamic design protocol that integrates ProtaBank with CPD software platforms, such as Triad, to produce protein designs informed by experimental data. Advanced query/search tools were developed to identify and retrieve data based on protein identity, local or global sequence similarity, and other criteria. This integration allows Triad to directly access this data to identify beneficial mutations (informing design parameters) and to create hybrid score functions that identify variants with good structural properties (using CPD score function terms) and good assay potential (using a data-based scoring term).

FIG. 12 illustrates an embodiment of a staged method for engineering proteins using AI and the Protabank database. Stage 1) Generation of potential sequences using various techniques from random mutation to computationally designed combinatorial libraries. Stage 2) Application of a previously trained machine learning model for prediction purposes. Stage 3) Evaluation of a large number of potential sequences for the desired properties of interest. Stage 4) Selection of high performing sequences, either individually or as a library of sequences; for example, as an optimized degenerate codon library. Stage 5) Validation of designed sequences using experimental assays. These new data points could now be combined with prior existing data to generate a more predictive machine learning model and the process iterated until the desired optimal protein sequence mutations are found.

### EXAMPLES

### Example 1

The database, deposition tools, and AI Platform have been currently implemented as shown in FIGS. 1, 2, 8, and 10. ProtaBank is currently the largest repository of protein sequence mutation data, containing 1584 studies on 691 unique proteins with over 5 million data points that associate mutant protein sequences with their measured properties. Statics for ProtaBank are given in Table 2.

TABLE 2
Table 2. ProtaBank database statistics.

| Statistic | Count |
|---|---|
| Studies | 1,584 |
| Protein variants | 1,581,675 |
| Data points | 5,236,368 |
| Unique proteins | 691 |
| Assays | 14,738 |

### Example 2

The following case studies demonstrate ProtaBank's utility in searching, analyzing and interpreting PE data.

Tool 1: Compare data for a protein sequence

Before beginning any PE study, a review of existing literature on the protein of interest provides a useful reference point. Therefore, a simple but important application of ProtaBank is to identify and compare previously measured properties of a given sequence. Because ProtaBank stores the full sequence information for each mutant, a query on a specified protein sequence retrieves all the relevant data for that sequence, even if the starting sequences were different. In this example, ProtaBank's “Compare data for a sequence” tool was used to search for data on the wild-type sequence of the β1 domain of Streptococcal protein G (Gβ1): MTYKLILNGKTLKGETTTEAVDAATAEKVFKQYANDNGVDGEWTYDDATKTFT VTE. ProtaBank returns a sortable and searchable table listing all the data for the specified search sequence, including all the properties, assays, results, units, and titles of the associated studies. This data table is then searched for “Gibbs free energy” to just show the data in which ΔGs were measured. The ΔG search shows five experimentally measured values for ΔG of unfolding (ΔGᵤ) from five studies, 29-33 with values differing by up to 1.8 kcal/mol.

These differences could represent statistical variation in the measurement of this property. However, differences in assay techniques or conditions could also be responsible. ProtaBank provides links in the data table so that a user can quickly view the details for each assay. For example, a careful examination of assay details shows that an important difference between the assays was the pH used for denaturation; the temperature was 25° C. for all the measurements except one (see Table 3). Different techniques were also used (chemical vs. thermal denaturation), but these gave similar results when the temperature and pH were similar. These results suggest that the pH and/or temperature can have a notable effect on ΔGᵤ. Thus, in order to make meaningful comparisons of engineered mutants relative to the wild type, it is clearly important to select the results with the most closely matched experimental conditions. By facilitating these types of comparisons, ProtaBank provides context for the results in each study, reveals assay parameters that can impact the results, and enables an informed evaluation of results obtained under different assay conditions.

TABLE 3
Assay Details Help Explain Differences in
ΔGᵤ Results for wild-type Gβ1

| | | ΔGᵤ | | | |
|---|---|---|---|---|---|
| | | (kcal/ | | T | |
| Study | Reference | mol)^{a} | Technique^{b} | (° C.)^{c} | pH |
| 57 | Choi and | 5.9^{d} | Thermal denaturation, | 25 | 5.5 |
| | Mayo, 2006 | | circular dichroism | | |
| 61 | Gronenborn | 5.6 | GdmCl denaturation, | 25 | 5.4 |
| | et al., 1996 | | fluorescence | | |
| 72 | Frank et al., | 4.1 | Urea denaturation, | 25 | 2.0 |
| | 1995 | | fluorescence | | |
| 74 | Kuszewski | 4.8 | GdmCl denaturation, | 5 | 4.0 |
| | et al., 1994 | | fluorescence | | |
| 171 | Davey et al., | 4.1 | GdmCl denaturation, | 25 | —^{e} |
| | 2017 | | fluorescence | | |

^{a}ΔGᵤ, Gibbs free energy of unfolding;
^{b}GdmCl, guanidinium chloride;
^{c}T, temperature;
^{d}Value was −5.9 for ΔG of folding; ΔGᵤ is therefore opposite in sign (5.9);
^{e}pH not reported.

For theoretical and computational scientists, ProtaBank permits easy access to data sets that can be used to benchmark, test, and improve predictive methods. For example, the experimental results provided in this example could be used to test theoretical methods aimed at predicting the effect of pH and/or temperature on a protein's stability based on how many ionizable side chains it contains.

Tool 2: Identify and Analyze Sequence Mutations

Protein engineers are typically not only interested in the data reported for a given sequence, but in the data reported for closely related sequences. By comparing results between a sequence and its mutants, the effects of mutation at a given position can be determined. The knowledge gained can then be used to guide the selection of positions and mutations in future engineering efforts. ProtaBank's “Identify and analyze sequence mutations” tool is used to retrieve all the studies and assays containing data for sequences closely related to wild-type Gβ1. After entering the sequence in the search box, a BLAST search is performed to identify all related mutant sequences. The BLAST search currently identifies ˜1.3 million sequences in ProtaBank that are closely related to wild-type Gβ1. Summary information is displayed in a mutant distribution heat map and a histogram showing the distribution of the number of mismatches (FIG. 4). The heat map [FIG. 4(A)] shows the number of sequences containing a mutation to a given amino acid at a given position; the wild-type residue for each position is shown in white. The heat map reveals that the T2Q mutation occurs most frequently and that mutants at positions 39, 40, 41, and 54 represent a large number of all the mutants identified. The T2Q mutation is often included in studies of Gβ1 to prevent cleavage of the N-terminal methionine by post-processing enzymes, and the preponderance of data for positions 39, 40, 41, and 54 is explained by a study that examined all possible combinations of mutations at these four positions, a total of 160,000 (204) variants. The histogram [FIG. 4(B)] shows the number of sequences found at each mismatch level, where the number of mismatches is the number of mutations needed to go from a given mutant sequence to the search sequence. In this example, most of the sequences are two or three mutations away from the search sequence. These plots show information that can help users determine which positions and mutations have already been studied and which new ones they might want to consider in future work.

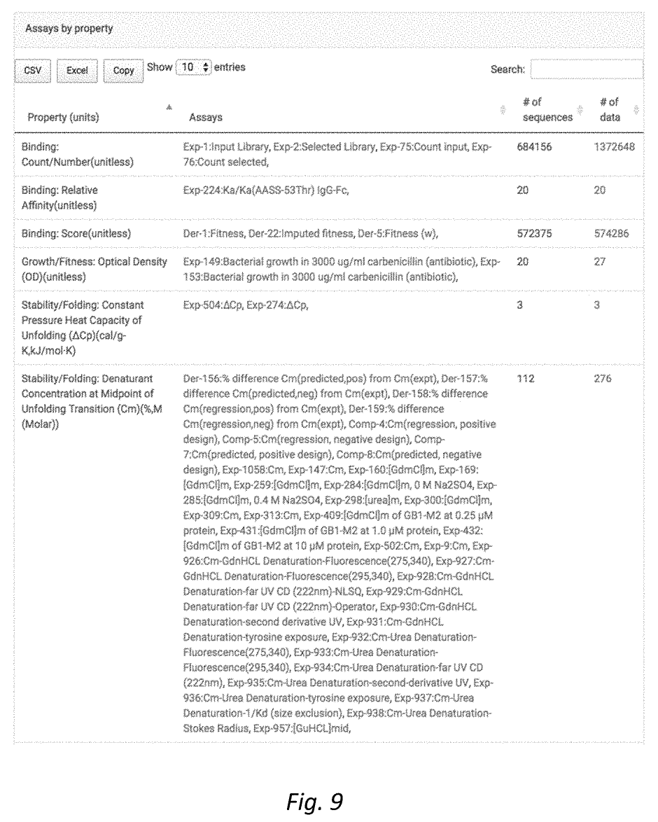

An “Assays by property” table is also displayed that lists all the assays containing data for a related mutant sequence, grouped by the protein property measured (FIG. 9). For each property, the table identifies all the individual assays, number of unique sequences, and total number of data points. Links to each of the assays provide quick and intuitive access to assay details. Each of the data sets can be viewed via the # of data points link, which opens up a table displaying the results for that data set. This information can be downloaded as a CSV or Excel file.

Tool 3: Compare Assays

Here the “Compare assay” is used in conjunction with the “Identify and analyze sequence mutations” tool to perform further analyses on the closely related Gβ1 sequences retrieved above.

Plot One Property Vs. Another

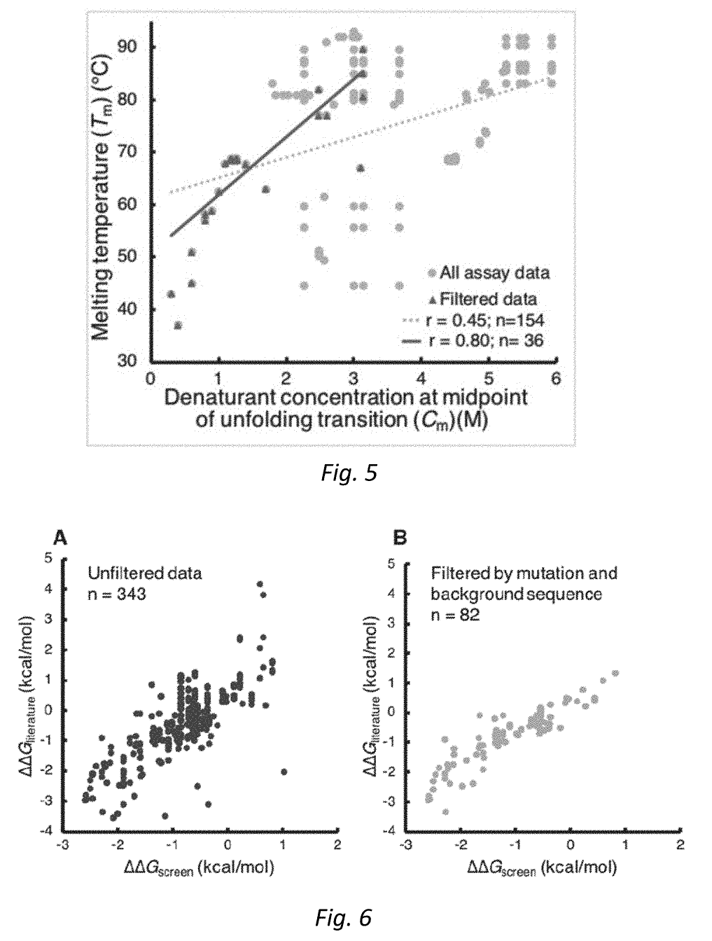

For any two measured properties, users can plot one property vs. another to show how these properties are correlated. ProtaBank automatically performs the unit conversions required to plot the data on the same set of axes. In FIG. 5, for example, two measures of stability are compared: Tm and the denaturant concentration at the midpoint of the unfolding transition (Cm) for all the closely related sequences of Gβ1 retrieved in the BLAST search above (see Tool 2). A plot of all the Gβ1 mutant sequences for which both properties were measured (circles) shows a moderate correlation (r=0.45) between these two properties, which could be explained by the fact that this comparison does not take differences in assay conditions into account. ProtaBank facilitates comparison of assay details by providing links to each of the assays listed in the “Assays by property” table (FIG. 9). If Tm vs. Cm for the Gβ1 mutants is replotted using only data measured under similar assay conditions (e.g., pH, temperature, and denaturant), a very strong correlation is observed (r=0.80) (FIG. 5, triangles).

Compare Assay Results

The “Compare assay to others by mutation” feature allows all the input mutants for one assay to be searched for and compared to a given group of assays. ProtaBank automates the time-consuming task of manually identifying relevant literature results, converting the data to the same set of units, and displaying pertinent assay and background sequence information. All the results can then be further sorted and filtered by background sequence, mutation, or study. This feature can be used to compare new ΔΔG measurements to existing biochemical measurements of ΔΔG. ProtaBank search tools were used to reproduce data from a study by Olson et al. in which Gβ1 fitness values were used to predict the change in stability upon point mutation. The ΔΔG predictor values (ΔΔG_{screen}) were plotted against experimental ΔΔG values reported in the literature (ΔΔGₗᵢₜₑᵣₐₜᵤᵣₑ). First, a “Compare assay to others by mutation” was done on the closely related sequences of wild-type Gβ1; this search identified hundreds of mutant sequence pairs in ProtaBank [FIG. 6(A)]. Then these results were filtered to the set of 10 background sequences and single point mutants listed in the Olson et al. study [FIG. 6(B)]. The filtered results match the data in their paper exactly except for one point—the mutant cited as I6L29 is actually a double mutant (I6L+T2Q) and was therefore excluded in the single mutant results. Note that ProtaBank identifies ˜260 additional data points. This feature makes it easy to compare the results for the set of mutants in a given assay to those from any other group of assays (the properties measured can be the same or different). This allows one to see if new assay data is consistent with previously observed trends. It can also be used to identify protein properties that are well correlated with a particular assay.

Tool 4: Visualize the Relationship Between Mutations and Protein Structure

This tool maps the effect of single mutations onto the crystal structure of the protein. By visualizing the data in this way, the functional significance of structural features becomes more obvious than when viewed in a table or chart.

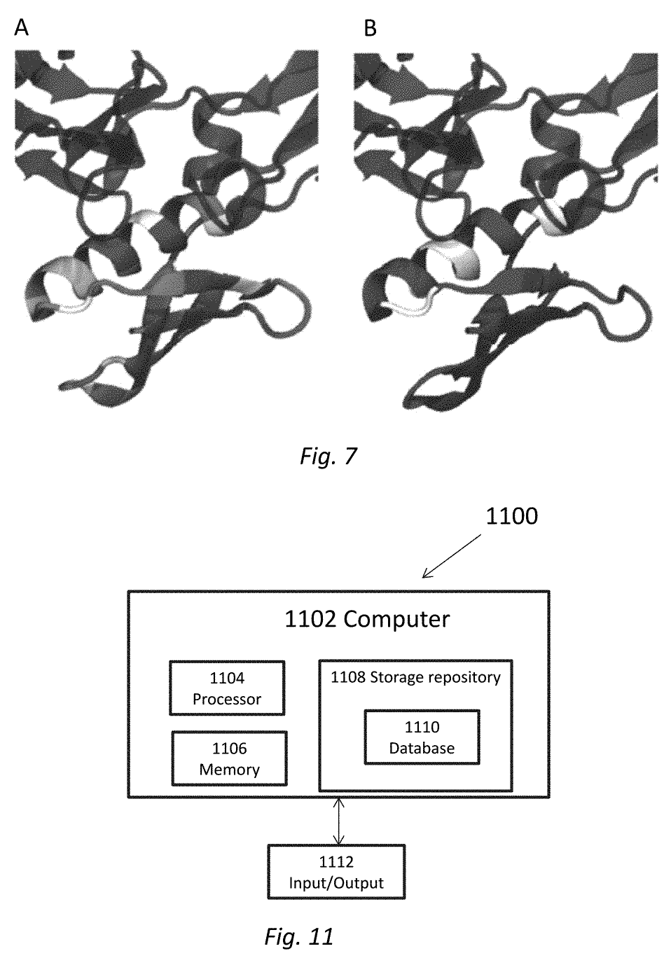

ProtaBank allows the user to save the data values from the selected color scheme in the occupancy column of the PDB file so that other modeling or visualization software can be used. In this example, visual molecular dynamics (VIVID) software was used to make the images shown in FIG. 7. Two views of Gβ1 bound to the Fc domain (PDB ID: 1FCC)44 are displayed. On the left, the Gβ1 backbone is shaded by median deviation from the wild-type value. On the right, the backbone is shaded by proximity to the binding site. Most of the residues near the binding site also show large median deviations from the wild-type value. These results are understandable given that the study employed a selection assay based on Fc binding. The structural analysis thus helps explain why these residues are particularly sensitive to mutation and suggests that the observed sensitivity is likely due to disruption of the binding site rather than a destabilization of the Gβ1 fold.

ProtaBank provides more advanced integration with protein structural data to allow for data selection and filtering on structural properties and to allow for computational predictions based on structural and sequence information; and incorporates computational methods to predict the effect of mutations on protein properties such as stability, binding, and activity.

### Example 3

FIG. 3A shows a screenshot of the web interface in which the “Browse submitted studies” tool was used to filter studies by protein name (“ubiquitin”). This search returns a sortable table containing all studies with ubiquitin in the protein name or study title. Clicking on the study ID at the left brings up the analysis page for that study.

FIG. 3B shows a screenshot in which study analysis tools were used to visualize mutational data for a study on β-lactamase which includes a protein visualizer in which mutational results are mapped onto the protein structure according to the selected color scheme. Here, Leu57 is mutated and the single mutant data for that residue is displayed in the tables below and to the right. Leu57 was mutated to His, Ile, and Pro, resulting in scores of −1.66, 0.25, and −5.32, respectively (mean=−2.24).

The visualizer is based on PV, an open-source javascript protein viewer (https://biasmv.github.io/pv/index.html) that was extended to allow mutations to be represented on the 3D structure using different color schemes. These include shading by secondary structure, gradient, minimum, maximum, median, mean, proportion above a reference value, and median deviation from a reference value. In the study depicted here, Jacquier et al. investigated the effects of mutations on TEM-1 β-lactamase activity by computing the amoxicillin minimum inhibitory concentration (MIC) score for ˜990 point mutants. FIG. 3(B) shows the crystal structure of TEM-1 (PDB ID: 1BTL) displayed with the backbone shaded by the MIC score. In this case, the median deviation from the wild-type value is shown. Pointing the cursor at a specific residue highlights it in and displays additional information for that residue in the tables below and to the right.

### Example 4

ML protocols were developed that use ProtaBank data to predict the effect of mutations on protein properties. The protocols were implemented with a Python package that communicates with ProtaBank and implemented basic sequence-based encodings to transform ProtaBank data into a form amenable for ML, and generated and applied neural network predictors to model the data and suggest variant sequences to test.

FIG. 12 illustrates a ProtaBank's AI Platform method used to train a model that predicts solubility and fitness for TEM-1 β-lactamase mutants based on deep mutational scanning data deposited in ProtaBank. The input tensor encodes amino acid sequences using physical properties. The output tensor holds the associated solubility and fitness assay data from ProtaBank. The training input/output tensors are used to train a multitask neural network with 4 dense layers.

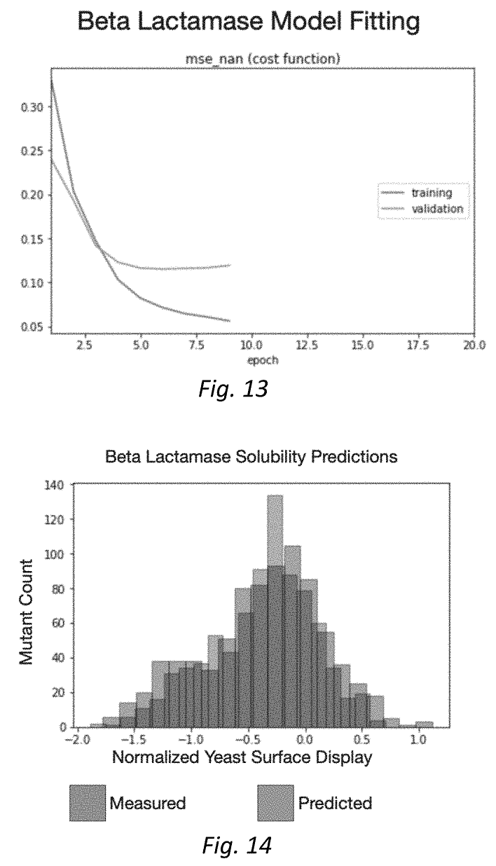

FIG. 13 shows a graph of model performance (as measured by mean squared error (MSE)) over the course of the training process using an 80/20 training validation split. At the end of the training process, the combined validation MSE of ˜0.13 corresponds to an MSE of ˜0.09 for TEM-1 solubility prediction and ˜0.06 for TEM-1 fitness prediction.

FIG. 14 shows that the distribution of predicted solubility values (validation set) closely matches the distribution of experimentally measured values.

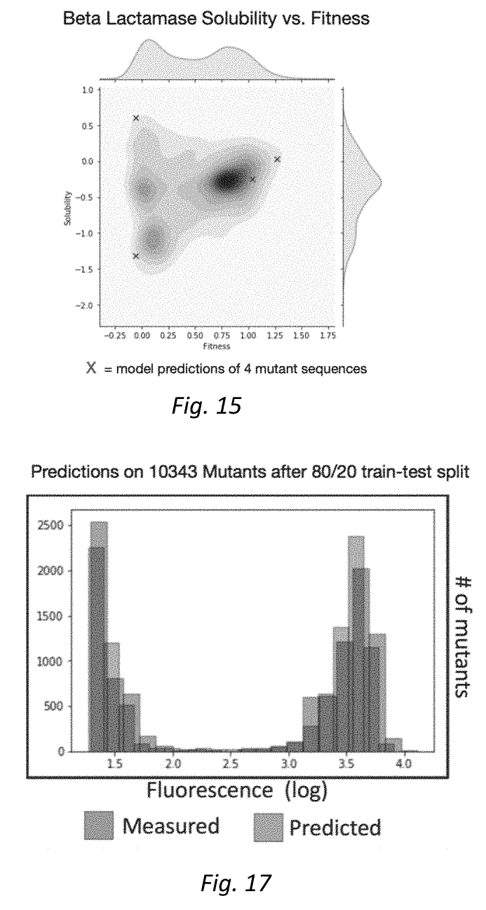

FIG. 15 shows the solubility/fitness landscape for mutants in ProtaBank. The trained model can be applied to unseen trial sequences to predict where they fall on the solubility/fitness landscape.

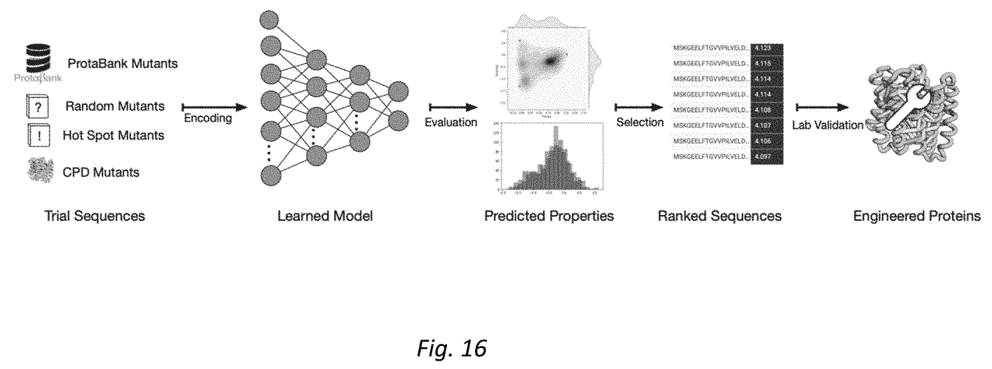

FIG. 16 shows how the trained model can be applied to trial sequences to predict protein properties. A ranking or selection algorithm can be used to select sequences or design libraries of sequences for lab validation.

### Example 5

In FIG. 17, ProtaBank's AI Platform predictions for fluorescence of green fluorescent protein (GFP) mutants using a neural network with 4 dense layers after encoding the structural features shown in the input tensor from deep mutational scanning data deposited in ProtaBank. GFP mutants' log fluorescence was predicted with a mean squared error of ˜0.1, showing the utility of ProtaBank data for protein engineering using ML.

Although embodiments described herein are made with reference to example embodiments, it should be appreciated by those skilled in the art that various modifications are well within the scope and spirit of this disclosure. Those skilled in the art will appreciate that the example embodiments described herein are not limited to any specifically discussed application and that the embodiments described herein are illustrative and not restrictive. From the description of the example embodiments, equivalents of the elements shown therein will suggest themselves to those skilled in the art, and ways of constructing other embodiments using the present disclosure will suggest themselves to practitioners of the art. Therefore, the scope of the example embodiments is not limited herein.

### REFERENCES

Gronenborn A M, Frank M K, Clore G M (1996) Core mutants of the immunoglobulin binding domain of streptococcal protein G: stability and structural integrity. FEBS Lett 398:312-316.
Frank M K, Clore G M, Gronenborn A M (1995) Structural and dynamic characterization of the urea denatured state of the immunoglobulin binding domain of streptococcal protein G by multidimensional heteronuclear NMR spectroscopy. Protein Sci 4:2605-2615.
Kuszewski J, Clore G M, Gronenborn A M (1994) Fast folding of a prototypic polypeptide: the immunoglobulin binding domain of streptococcal protein G. Protein Sci 3:1945-1952.
Choi E J, Mayo S L (2006) Generation and analysis of proline mutants in protein G. Protein Eng Des Sel 19:285-289.
Davey J A, Damry A M, Goto N K, Chica R A (2017) Rational design of proteins that exchange on functional timescales. Nat Chem Biol 13:1280-1285.
Wang, C. Y., Chang, P. M., Ary, M. L., Allen, B. D., Chica, R. A., Mayo, S. L. et al., Olafson, B. D. ProtaBank: A repository for protein design and engineering data. Protein Sci 27, 1113-1124 (2018). The whole of which is incorporated herein by reference.
Jacquier H, Birgy A, Le Nagard H, Mechulam Y, Schmitt E, Glodt J, Bercot B, Petit E, Poulain J, Barnaud G, Gros P-A, Tenaillon O (2013) Capturing the mutational landscape of the beta-lactamase TEM-1. Proc Natl Acad Sci USA 110:13067-13072.

What is claimed is:

1. A system for engineering proteins based on mutational, the system comprising:
a processor;
a storage repository comprising:
a database comprising:
a plurality of full length mutant protein sequences, each full length mutant protein sequence comprising a string representing an amino acid sequence; and
a plurality of characteristic data sets, wherein each characteristic data set has an associated full length mutant protein sequence from the plurality of full length mutant protein sequences and wherein the characteristic data set includes data from assays done with a protein of the associated full length mutant protein sequence;
an AI Platform comprising:
computer executable instructions for execution by the processor, the computer executable instructions performing steps comprising:
generating an AI training set comprising one or more of the full length mutant protein sequences from the plurality of full length mutant protein sequences in the database;
encoding an input tensor comprising the amino acid sequences of the plurality of full length mutant protein sequences from the AI training set;
encoding an output tensor comprising of one or more of the plurality of characteristic data associated with the plurality of full length mutant protein sequences from the AI training set; and
generating a machine learning model using a machine learning framework configured to input the input tensor and the output tensor, and to generate the machine learning model.

2. The system of claim 1, wherein encoding the input tensor comprises encoding individual amino acid characteristics, partial sequences characteristics, or local behavior characteristics.

3. The system of claim 2, wherein the data from assays comprise experimental assay type, numerical value obtained for the assay, and units associated with the numerical value.

4. The system of claim 1, wherein the characteristic data sets additionally comprise protein structure data.

5. The system of claim 4, wherein encoding the input tensor depends on the protein structure data.

6. The system of claim 1, wherein the input tensors comprises one or more of charge, hydrophobicity, and volume associated with amino acids in the amino acid sequences of the plurality of full length mutant protein sequences from the AI training set.

7. The system of claim 1, wherein the machine learning framework comprises one or more of a neural network, genetic algorithm, decision tree, gradient boosting, and support vector machines.

8. The system of claim 1, wherein the computer executable instructions further comprise instructions for;
receiving a protein identifier and protein functional data;
matching the identifier to one or more full length mutant protein sequences stored in the database;
creating the AI training set with the matched full length mutant protein sequences;
generating a plurality of synthetic sequences;
applying the machine learning model to the plurality of synthetic sequences to generate predicted protein functional data for each synthetic sequence; and
outputting one or more of the synthetic sequences and associated predicted protein functional data.

9. The system of claim 8, further comprising:
generating a subset of synthetic sequences in which the predicted protein functional data is within a predetermined range of the received protein functional data.

10. The system of claim 9, wherein the synthetic sequences are generated by random mutation or by a computationally designed combinatorial library.

11. The system of claim 9, wherein the received protein functional data comprises one or more of the following: Activity, Catalytic efficiency (k_{cat}/Kₘ), Catalytic rate constant (k_{cat}), Count/Number, EC50, Energy, Enrichment, Epistasis, Fitness, IC50, Inhibition constant (Kᵢ), Maximal rate (Vₘₐₓ), Michaelis constant (Kₘ), Relative activity, Specific activity, Association constant (Kₐ), Binding affinity, Count/Number, Dissociation constant (K_{d}), ELISA, Energy, Enrichment, Enthalpy of binding (ΔH), Entropy of binding (ΔS), Epistasis, Fitness, Frequency of occurrence, Gibbs free energy of binding (ΔG), Inhibition constant (Kᵢ), Rate constant of association (kₒₙ), Rate constant of dissociation (k_{off}), Concentration, Energy, Enrichment, Frequency of occurrence, Minimum inhibitory concentration (MIC), Yield, Antimicrobial resistance, Energy, Enrichment, Frequency of occurrence, Optical density (OD), Bioavailability, EC50, Half-life (tin), IC50, Immunogenicity, Toxicity, Concentration, Energy, Fractional increase in solubility, Insoluble fraction, Oligomerization state, Soluble fraction, Energy, Frequency of occurrence, Relative activity, Relative affinity, Relative k_{cat}, Relative k_{cat}/Kₘ, Relative K_{d}, Brightness, Emission wavelength (λₑₘ), Energy, Excitation wavelength (λₑₓ), Extinction coefficient, Fluorescence intensity, Maturation half-time, Photobleaching half-time, pKa, Quantum yield, Constant pressure heat capacity of unfolding (ΔCₚ), Count/Number, Denaturant concentration at midpoint of unfolding transition (Cₘ), Energy, Enthalpy of unfolding (ΔH), Entropy of unfolding (ΔS), Equilibrium constant (K), Gibbs free energy of folding/unfolding (ΔG), Melting temperature (Tₘ), Rate of folding (k_{F}), Rate of unfolding (k_{U}), Slope of chevron plot (m), Slope of the denaturant unfolding curve/cooperativity value (m), Temperature of maximum stability, Thermal tolerance, ß-Tanford value, and Φ-value.

12. The system of claim 9, wherein the protein identifier is a name or a full length protein sequence.

13. The system of claim 9, wherein matching comprises comparing the full length protein sequence of the protein identifier to full length mutant protein sequences in the database and returning a match when the sequences are at least 20%, 30%, 40%, 50%, 60%, 70%, 80%, 90%, 95%, or more than 99% similar.

14. The system of claim 1, wherein the characteristic data set comprises one or more of the following: Activity, Catalytic efficiency (k_{cat}/Kₘ), Catalytic rate constant (k_{cat}), Count/Number, EC50, Energy, Enrichment, Epistasis, Fitness, IC50, Inhibition constant (Kᵢ), Maximal rate (Vₘₐₓ), Michaelis constant (Kₘ), Relative activity, Specific activity, Association constant (Kₐ), Binding affinity, Count/Number, Dissociation constant (K_{d}), ELISA, Energy, Enrichment, Enthalpy of binding (ΔH), Entropy of binding (ΔS), Epistasis, Fitness, Frequency of occurrence, Gibbs free energy of binding (ΔG), Inhibition constant (Kᵢ), Rate constant of association (kₒₙ), Rate constant of dissociation (k_{off}), Concentration, Energy, Enrichment, Frequency of occurrence, Minimum inhibitory concentration (MIC), Yield, Antimicrobial resistance, Energy, Enrichment, Frequency of occurrence, Optical density (OD), Bioavailability, EC50, Half-life (tin), IC50, Immunogenicity, Toxicity, Concentration, Energy, Fractional increase in solubility, Insoluble fraction, Oligomerization state, Soluble fraction, Energy, Frequency of occurrence, Relative activity, Relative affinity, Relative k_{cat}, Relative k_{cat}/Kₘ, Relative K_{d}, Brightness, Emission wavelength (λₑₘ), Energy, Excitation wavelength (λₑₓ), Extinction coefficient, Fluorescence intensity, Maturation half-time, Photobleaching half-time, pKa, Quantum yield, Constant pressure heat capacity of unfolding (ΔCₚ), Count/Number, Denaturant concentration at midpoint of unfolding transition (Cₘ), Energy, Enthalpy of unfolding (ΔH), Entropy of unfolding (ΔS), Equilibrium constant (K), Gibbs free energy of folding/unfolding (ΔG), Melting temperature (Tₘ), Rate of folding (k_{F}), Rate of unfolding (k_{U}), Slope of chevron plot (m), Slope of the denaturant unfolding curve/cooperativity value (m), Temperature of maximum stability, Thermal tolerance, ß-Tanford value, and Φ-value.

15. A method of engineering proteins performed by a computing system comprising a processor executing instructions stored in a non-transitory computer-readable medium, the method comprising:
storing a plurality of full length mutant protein sequences, each full length mutant protein sequence comprising a string representing an amino acid sequence;
storing a plurality of characteristic data sets, wherein each characteristic data set has an associated full length mutant protein sequence from the plurality of full length mutant protein sequences and wherein the characteristic data set includes data from assays done with a protein of the associated full length mutant protein sequence;
receiving a protein identifier and protein functional data;
matching the protein identifier to one or more full length mutant protein sequences stored in the database;
generating an AI training set with the matching full length mutant protein sequences;
training a machine learning model using the AI training dataset;
employing the machine learning model to design one or more synthetic protein sequences and calculate each synthetic proteins predicted functional data; and
outputting the one or more synthetic protein sequences and predicted functional data.

16. The method of claim 15, wherein the data from assays comprises one or more of experimental assay type, numerical value obtained for the assay, units associated with the numerical value, and derived values dependent on other experimental values.

17. The method of claim 15, wherein the machine learning model comprises one or more of a neural network, genetic algorithm, decision tree, gradient boosting, and support vector machines.

18. The method of claim 15, wherein matching comprises comparing the full length protein sequence of the protein identifier to the full length mutant protein sequences in the database and returning a match when the sequences are at least 20%, 30%, 40%, 50%, 60%, 70%, 80%, 90%, 95%, or more than 99% similar.

19. The method of claim 15, wherein the characteristic data set, the protein functional data, or both comprises one or more of the following: Activity, Catalytic efficiency (k_{cat}/Kₘ), Catalytic rate constant (k_{cat}), Count/Number, EC50, Energy, Enrichment, Epistasis, Fitness, IC50, Inhibition constant (Kᵢ), Maximal rate (Vₘₐₓ), Michaelis constant (Kₘ), Relative activity, Specific activity, Association constant (Kₐ), Binding affinity, Count/Number, Dissociation constant (K_{d}), ELISA, Energy, Enrichment, Enthalpy of binding (ΔH), Entropy of binding (ΔS), Epistasis, Fitness, Frequency of occurrence, Gibbs free energy of binding (ΔG), Inhibition constant (Kᵢ), Rate constant of association (kₒₙ), Rate constant of dissociation (k_{off}), Concentration, Energy, Enrichment, Frequency of occurrence, Minimum inhibitory concentration (MIC), Yield, Antimicrobial resistance, Energy, Enrichment, Frequency of occurrence, Optical density (OD), Bioavailability, EC50, Half-life (t_{1/2}), IC50, Immunogenicity, Toxicity, Concentration, Energy, Fractional increase in solubility, Insoluble fraction, Oligomerization state, Soluble fraction, Energy, Frequency of occurrence, Relative activity, Relative affinity, Relative k_{cat}, Relative k_{cat}/Kₘ, Relative K_{d}, Brightness, Emission wavelength (λₑₘ), Energy, Excitation wavelength (λₑₓ), Extinction coefficient, Fluorescence intensity, Maturation half-time, Photobleaching half-time, pKa, Quantum yield, Constant pressure heat capacity of unfolding (ΔCₚ), Count/Number, Denaturant concentration at midpoint of unfolding transition (Cₘ), Energy, Enthalpy of unfolding (ΔH), Entropy of unfolding (ΔS), Equilibrium constant (K), Gibbs free energy of folding/unfolding (ΔG), Melting temperature (Tₘ), Rate of folding (k_{F}), Rate of unfolding (k_{U}), Slope of chevron plot (m), Slope of the denaturant unfolding curve/cooperativity value (m), Temperature of maximum stability, Thermal tolerance, ß-Tanford value, and Φ-value.
